(19) United States
(12) Patent Application Publication
(10) Pub. No.: US 2010/0256381 A1
(43) Pub. Date: Oct. 7, 2010

(54) PROCESS FOR PRODUCING CISATRACURIUM AND ASSOCIATED INTERMEDIATES

(75) Inventors: Oded Arad, Rehovot (IL); Vladimir Naddaka, Petach Tikva (IL); Eyal Klopfer, Tel Aviv (IL); Shady Saeed, Haifa (IL); Ofer Sharon, Petach Tikva (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: CHEMAGIS LTD., Bnei Brak (IL)

(21) Appl. No.: 12/667,634

(22) PCT Filed: May 1, 2008
(86) PCT No.: PCT/IL08/00590

§ 371 Date: May 24, 2010

Related U.S. Application Data
(60) Provisional application No. 60/948,621, filed on Jul. 9, 2007.

(51) Int. Cl. C07D 401/12

(52) U.S. Cl. 546/140

(57) ABSTRACT

The present invention provides a process of producing cisatracurium compounds, e.g., cisatracurium besylate, from isoquinolinium salts of the structural formula (VIIA) wherein X⁻ is an anion and R is H or a C₁-C₆ alkyl, or an activated form of the carboxylic acid with 1,5-pentanediol to form a cisatracurium salt, optionally via an intermediate compound (VIII). The cisatracurium compounds can be purified using simple techniques to afford pure cisatracurium besylate without the need for HPLC purification.

### CROSS-REFERENCE TO RELATED APPLICATIONS

This patent application claims the benefit of U.S. Provisional Patent Application No. 60/948,621, filed Jul. 9, 2007, which is incorporated by reference.

### TECHNICAL FIELD

The present invention relates to organic chemistry and more particularly to preparation of novel isoquinolinium compounds and their use in the synthesis of cisatracurium compounds.

### BACKGROUND OF THE INVENTION

Cisatracurium besylate has the chemical name (1R,1′R,2R,2′R)-2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methyl-isoquinolinium dibenzenesulfonate and is represented by the structural formula (I) below:

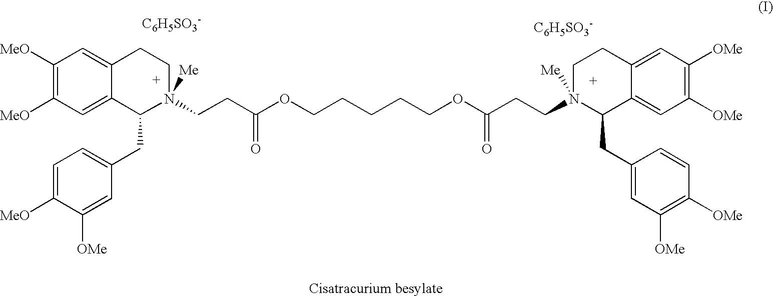

Cisatracurium besylate is the dibenzenesulfonate salt of 1R-cis,1′R-cis isomer of atracurium besylate. The atracurium compound has four chiral centers, which should theoretically allow for 16 possible isomers. Due to the symmetry of the molecule the number of possible isomers is reduced to 10. See, e.g., J. B. Stenlake et al. in “Biodegradable neuromuscular blocking agents,” Eur. J. Med. Chem.—Chem. Ther., vol. 19, issue 5, pp. 441-450 (1984).

Cisatracurium besylate is a nondepolarizing neuromuscular blocking agent indicated for inpatients and outpatients as an adjunct to general anesthesia, to facilitate tracheal intubation, and to provide skeletal muscle relaxation during surgery or mechanical ventilation in the Intensive Care Unit (ICU). Cisatracurium besylate possesses an activity that is superior to atracurium besylate, with significantly less side effects.

Cisatracurium besylate is marketed in the United States, Europe and other countries by GSK and Abbott Laboratories under the trade name Nimbex®. Nimbex® is a sterile, non-pyrogenic aqueous solution that is adjusted to pH 3.25 to 3.65 with benzenesulfonic acid. The drug is provided in 2.5 ml, 5 ml and 10 ml ampules having a strength of 2 mg/ml cisatracurium besylate. A 30 ml vial containing 5 mg/ml cisatracurium besylate is also available.

In formulation, Nimbex®, slowly loses potency with time at a rate of approximately 5% per year under refrigeration (5° C.). Nimbex should be refrigerated at 2° to 8° C. (36° to 46° F.) to preserve potency. The rate of loss in potency increases to approximately 5% per month at 25° C. (77° F.).

Atracurium besylate, also known as 2,2′-[1,5-pentanediylbis[oxy(3-oxo-3,1-propanediyl)]]bis[1-[(3,4-dimethoxyphenyl)methyl]-1,2,3,4-tetrahydro-6,7-dimethoxy-2-methyl-isoquinolinium dibenzenesulfonate (it is a mixture of isomers), is disclosed in U.S. Pat. No. 4,179,507 (hereinafter U.S. '507). U.S. '507 describes a series of bis veratryl isoquinolinium quaternary ammonium salts, including Atracurium besylate. U.S. '507 describes synthesizing atracurium besylate by a process that involves coupling (±)-tetrahydro-papaverine base (compound II) with 1,5-pentamethylene diacrylate (compound III) and treating the resulting tertiary amine base with oxalic acid to produce N,N′-4,10-dioxa-3,11-dioxotridecylene-1,13-bis-tetrahydropapaverine dioxalate (compound IV). This salt is converted to the free base (compound V), which is treated with methyl benzenesulfonate. The resulting product, atracurium besylate (compound VI), is precipitated and isolated. The process is illustrated below in Scheme 1.

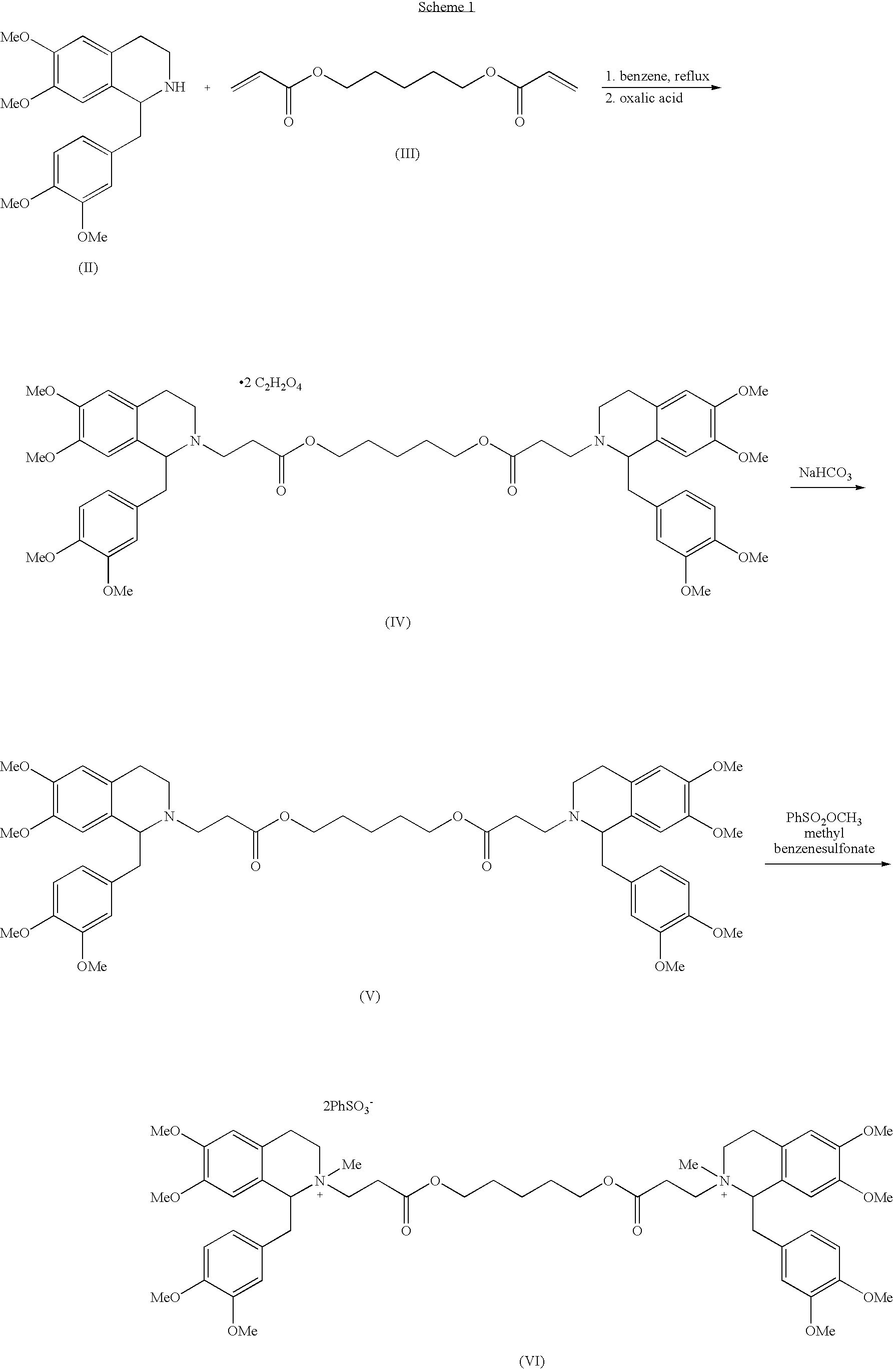

European application No. 0219616 (hereinafter E.P. '616) discloses the synthesis of atracurium chloride. E.P. '616 describes a process that involves coupling 1-[(3,4-dimethoxyphenyl)methyl]-3,4-dihydro-6,7-dimethoxy-2(1H)-isoquinolinepropanoic acid (compound VII) with 1,5-pentanediol in the presence of an acid to afford the diester (compound IX). The resulting diester is quaternized with methyl iodide to form atracurium iodide, which is then converted into atracurium chloride by means of anion exchange. The process is illustrated in below Scheme 2.

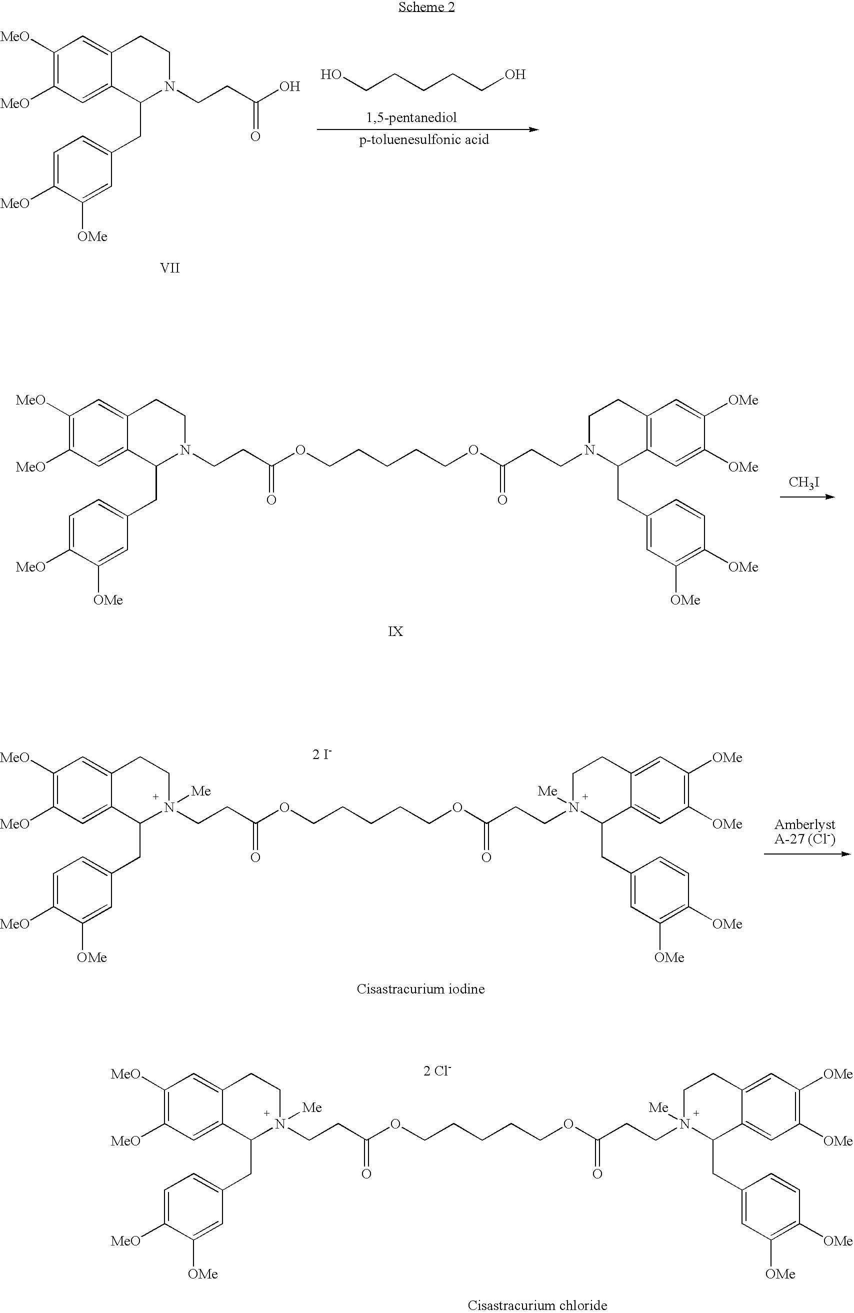

Cisatracurium besylate is disclosed in U.S. Pat. No. 5,453,510 (hereinafter U.S. '510). U.S. '510 describes the formation of (R)-tetrahydropapaverine (compound IIA) by converting compound (II) into a mixture of the R and S diastereoisomeric salts with the chiral amino acid N-acetyl-L-leucine and crystallizing from acetone to afford 97% (R)-tetrahydropapaverine-N-acetyl-L-leucinate and 3% (S)-tetrahydropapaverine-N-acetyl-L-leucinate, which is converted into (R)-tetrahydropapaverine base. The (R)-tetrahydro-papaverine is subsequently reacted with 1,5-pentamethylene diacrylate followed by oxalic acid to afford the dioxalate salt of (1R,1′R)-2,2′-(3,11-dioxo-4,10-dioxamidecamethylene)-bis-(1,2,3,4-tetrahydro-6,7-dimethoxy-1-veratrylisoquinoline) (i.e., an isomer of compound IV). Conversion of the dioxalate salt into the free base, followed by treatment with methyl benzenesulfonate, affords an aqueous solution of (1R,1′R)-atracurium besylate. Lyophilization results in a pale yellow solid that includes a mixture of three isomers, namely, 1R-cis,1′R-cis; 1R-cis,1′R-trans; 1R-trans,1′R-trans (hereinafter referred to as the “atracurium besylate mixture”) in a ratio of about 58:34:6 respectively. The atracurium besylate mixture is subjected to preparative HPLC column chromatography on silica using a mixture of dichloromethane, methanol and benzenesulfonic acid in the ratio of 4000:500:0.25 as the eluent. The fractions containing the required isomer are collected and further processed to afford cisatracurium besylate possessing an isomeric purity of about 99%.

The above procedure suffers from several disadvantages. A major problem in the procedure is attributable to the HPLC purification step. The need for HPLC purification is undesirable in a large-scale operation because only relatively small amounts of product can be purified at a time. The method is expensive, time-consuming and generates large quantities of waste solvents, which raises considerations with regard to safe disposal of the accumulated wastes. Another disadvantage of the above procedures is that cisatracurium besylate may be unstable in the eluent mixture used in the HPLC separation and, thus, can lead to the formation of decomposition products.

There is, therefore, a need for an improved process for the production of cisatracurium salts, e.g., cisatracurium besylate, and intermediates therefor, which avoids, where possible, the need for purifying the intermediates as well as the cisatracurium salt by column chromatography and can be scaled up to facilitate the large scale production of the cisatracurium salt (e.g., cisatracurium besylate). The present invention provides such a process.

### BRIEF SUMMARY OF THE INVENTION

The present invention provides a process for preparing cisatracurium salt e.g., cisatracurium besylate (I). In one embodiment, the process of the present invention includes:

(a) reacting a compound of formula (VITA):

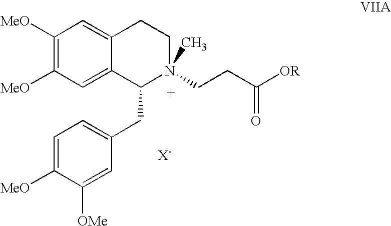

wherein X⁻ is an anion and R is H or a C₁-C₆ alkyl, with 1,5-pentanediol to form a cisatracurium salt, or reacting a compound of formula (VITA) with 1,5-pentanediol to form an intermediate compound of formula (VIII):

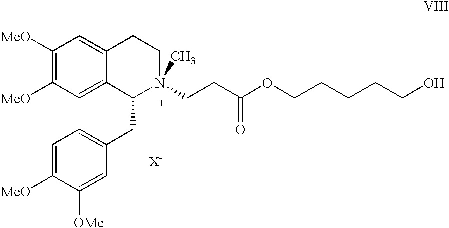

and reacting the compound of formula (VIII) with a compound of formula (VIIA) to form a cisatracurium salt, wherein X⁻ and R are as defined herein;

(b) optionally performing an ion exchange step, e.g., to form cisatracurium besylate (e.g., by exchanging the anion (X⁻) with another anion (X⁻) which is besylate);

(c) isolating the cisatracurium salt; and

(d) purifying the cisatracurium salt.

In some embodiments wherein R is H, the reaction can be carried out by direct acylation, that is, by refluxing Compound VIIA with 1,5-pentanediol in an organic solvent, e.g., dichloromethane or toluene, in the presence of an acid catalyst such as sulfuric acid or benzenesulfonic acid and by removal of water, e.g., by azeotropic distillation, using e.g., Dean-Stark apparatus, or by using molecular sieve.

In other embodiments wherein R is H, it may be desirable to activate the carboxylic acid for the subsequent conversion into an ester, e.g., wherein step (a) includes:

(i) reacting compound (VIIA), wherein R is H, with an activating agent, optionally in an organic solvent, to form a compound of the formula VIIB comprising an activated carboxylic group:

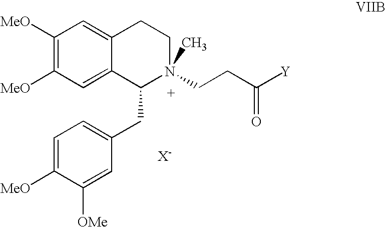

wherein Y is halogen, OR₁, or OCOR₁; (e.g., wherein R₁ is C₁-C₆ alkyl, e.g., methyl, ethyl, propyl, isopropyl, butyl, isobutyl, sec-butyl, tert-butyl, pentyl or hexyl); and

(ii) reacting the activated compound (VIIB) with 1,5-pentanediol to form a cisatracurium salt, or reacting the activated compound (VIIB) with 1,5-pentanediol to produce intermediate compound (VIII), and reacting compound (VIII) with compound (VIIB) to form a cisatracurium salt.

Step (b) can include contacting the cisatracurium salt with an ion exchange resin, e.g., an ion exchange resin carrying benzenesulfonate anions, to form cisatracurium besylate.

The isolation and purification steps can be carried out by any suitable separation or purification procedure such as, e.g., filtration, extraction, precipitation, crystallization, slurrying or any suitable combination of these procedures.

The process of the present invention preferably produces the cisatracurium salt, e.g., cisatracurium besylate, in at least about 95% purity, more preferably in at least about 98% purity, and most preferably in at least about 99.5% purity, as measured by HPLC. The process of the present invention preferably produces the cisatracurium salt, e.g., cisatracurium besylate, in an isomeric purity of at least about 97%, more preferably in an isomeric purity of at least about 99% and most preferably in an isomeric purity of at least about 99.5%, as measured by HPLC.

### DETAILED DESCRIPTION OF THE INVENTION

As used herein, the term “isomeric purity” refers to the area percent of the peak corresponding to the (1R-cis,1′R-cis) cisatracurium isomer relative to the total area percent of the (1R-cis,1′R-cis), (1R-cis,1′R-trans) and (1R-trans,1′R-trans) isomers as measured by HPLC.

The present invention provides a process for preparing cisatracurium besylate (I). In one embodiment, the process of the present invention includes:

(a) reacting a compound of formula (VIIA):

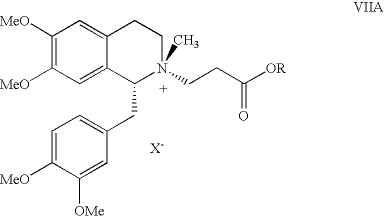

wherein X⁻ is iodide or besylate anion and R is H or a C₁-C₆ alkyl (methyl, ethyl, n-propyl, isopropyl, butyl, isobutyl, sec-butyl, tert-butyl, pentyl or hexyl), with 1,5-pentanediol to form a cisatracurium salt, or reacting a compound of formula (VIIA) with 1,5-pentanediol to form an intermediate compound of formula (VIII):

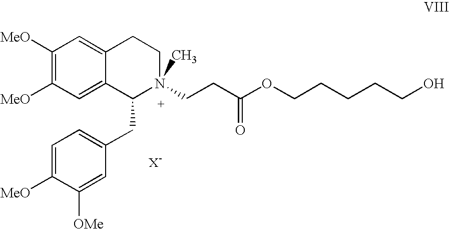

and reacting the compound of formula (VIII) with a compound of formula (VIIA) to form a cisatracurium salt, wherein X⁻ and R are as defined herein;

(b) optionally performing an ion exchange step, e.g., to form cisatracurium besylate (e.g., by exchanging the anion (X⁻), which is not besylate, with another anion (X), which is besylate);

(c) isolating the cisatracurium salt; and

(d) purifying the cisatracurium salt.

An exemplary process of the present invention is illustrated in Scheme 3 below.

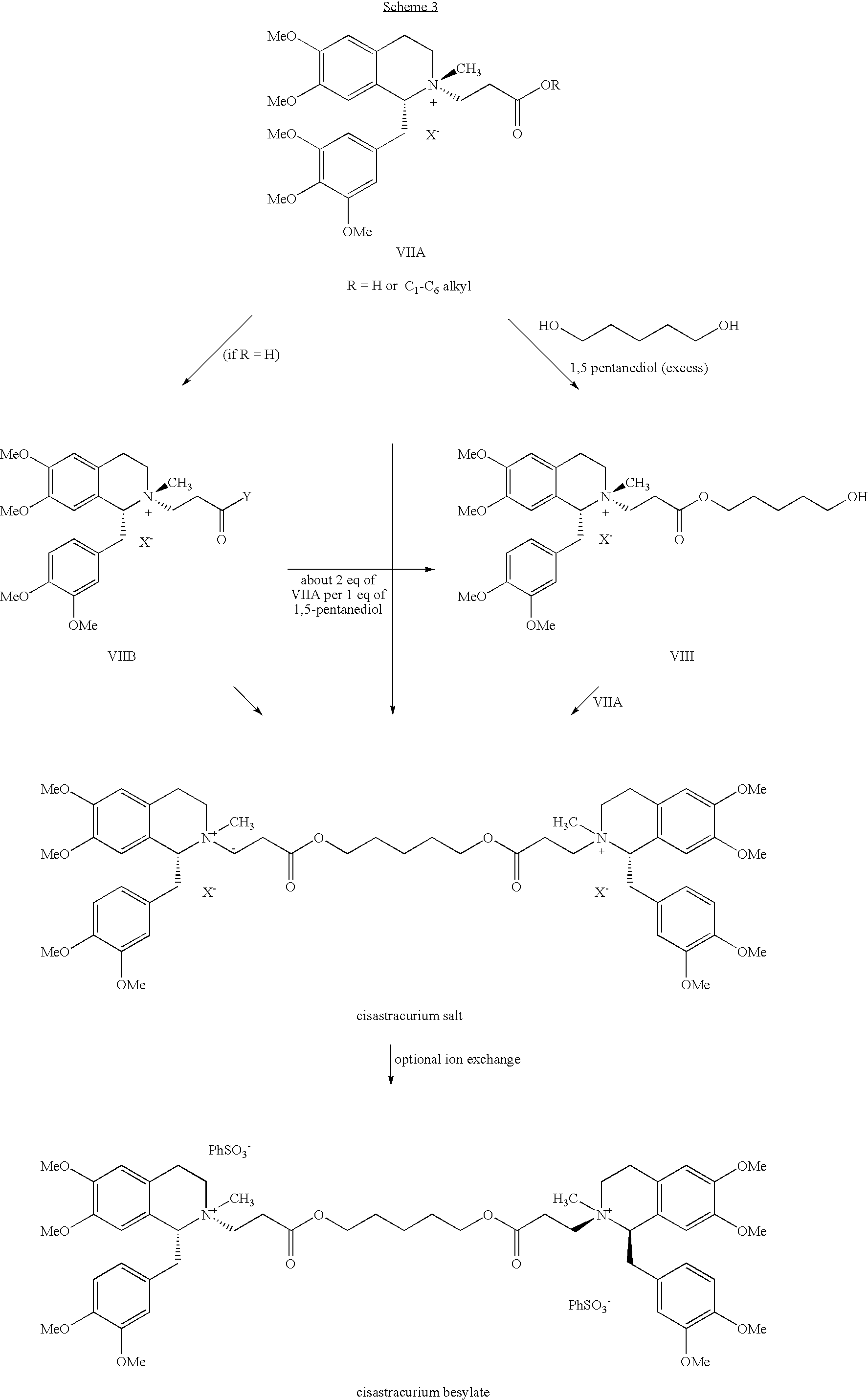

In a preferred embodiment, the anion X⁻ of formula (VIIA) is iodide or besylate, and R is methyl, ethyl, propyl, isopropyl, butyl, isobutyl, sec-butyl, tert-butyl, pentyl or hexyl.

In some embodiments wherein R is H, the reaction can be carried out by direct acylation, that is, by refluxing Compound VIIA with 1,5-pentanediol in an organic solvent, e.g., dichloromethane or toluene, in the presence of an acid catalyst such as benzenesulfonic acid or sulfuric acid and by removal of water, e.g., by azeotropic distillation using, e.g., a Dean-Stark apparatus or by using a drying agent such as molecular sieve, sodium sulfate, magnesium sulfate, calcium sulfate, and calcium chloride.

In other embodiments wherein R is H, it is necessary to activate the carboxylic acid for the subsequent conversion into an ester, e.g., wherein step (a) includes:

(i) reacting compound (VIIA), wherein R is H, with an activating agent, optionally in an organic solvent, to form a compound of the formula (VIIB) comprising an activated carboxylic group:

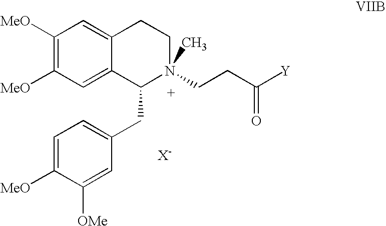

wherein Y is halogen, OR₁, or OCOR₁; (e.g., wherein R₁ is C₁-C₆ alkyl, e.g., methyl, ethyl, propyl, isopropyl, butyl, isobutyl, sec-butyl, tert-butyl, pentyl or hexyl); and

(ii) reacting the activated compound (VIIB) with 1,5-pentanediol to form a cisatracurium salt, or reacting the activated compound (VIIB) with 1,5-pentanediol to form intermediate compound (VIII) as described herein, and reacting compound (VIII) with activated compound (VIIB) (or with compound (VIIA) if desired) to form a cisatracurium salt.

A skilled artisan can expect that in cases when a starting material Compound VIIA possesses the opposite or S-configuration, the configuration of the end product will be retained as S-configuration to obtain the corresponding (1S-cis,1′S-cis), (1S-cis,1′S-trans) and (1S-trans,1′S-trans) isomers.

The activating agent can include, e.g., a chlorinating agent, an esterifying agent or an acid anhydride forming agent. In a preferred embodiment of the present invention, the activating agent is a chlorinating agent such as, for example, phosphorus oxychloride, thionyl chloride, phosphorous pentachloride or oxalyl chloride. Preferred chlorinating agents include oxalyl chloride and thionyl chloride.

The organic solvent used in the activation step can include, e.g., a chlorinated hydrocarbon or an aromatic hydrocarbon. In one embodiment, the carboxylic acid is activated in an organic solvent, which can include, e.g., dichloromethane, chloroform, 1,2-dichloroethane, xylenes, toluene or a mixture thereof. A preferred organic solvent for performing the activation step is dichloromethane.

The activation of the carboxylic group of compound (VIIA) is preferably performed at a reduced temperature, e.g., less than about 20° C.

In one embodiment, the chlorinating agent is added gradually (e.g., dropwise or in portions, depending on various factors such as, e.g., reaction scale) to the solution of compound (VIIA) at a reduced temperature, e.g., less than about 20° C., over a period of about 30 minutes.

The amount of chlorinating agent used in the activation step is preferably in the range of from about 1.0 to 2.0 equivalents relative to compound (VIIA). Preferably, the amount of chlorinating agent used in the activation step is in the range of about 1.0 to 1.2 equivalents relative to compound (VITA). In one embodiment, the activation step involves using about 1.1 equivalents of chlorinating agent relative to compound (VIIA).

In a preferred embodiment, 1,5-pentanediol is added to the activated compound gradually at a temperature of less than about 20° C. over a period of about 30 minutes.

The amount of 1,5-pentanediol used in the coupling reaction (whereby an ester of 1,5-pentanediol is formed) is preferably in the range of from about 0.5 equivalents to about 1.0 equivalents relative to compound (VIIA). In a preferred embodiment, the amount of 1,5-pentanediol used in the coupling reaction is in the range of from about equivalents 0.5 to about 0.7 equivalents relative to compound (VITA).

In accordance with the present invention, the coupling step (a) can include reacting compound (VIIB) with 1,5-pentanediol optionally in the presence of a catalyst, optionally in an organic solvent, to form the cisatracurium salt. Suitable catalysts include acidic catalysts such as, e.g., CaSO₄/benzenesulfonic acid, NaHSO₄.SiO₂, Amberlyst®15 (a sulfonic acid resin based on cross linked styrene-divinylbenzene copolymers) or a mixture of benzenesulfonic acid and silica gel of pH 3.0-5.0. NaHSO₄.SiO₂ is a heterogeneous acidic catalyst that includes sodium hydrogen sulfate supported on silica gel. Preferred acidic catalysts include CaSO₄/benzenesulfonic acid and NaHSO₄.SiO₂.

Organic solvents that can be used in the coupling reaction can include one or more chlorinated hydrocarbons, one or more aromatic hydrocarbons and mixtures thereof. Preferred organic solvents for performing the coupling reaction include dichloromethane, chloroform, 1,2-dichloroethane, xylenes, toluene and mixtures thereof. In one embodiment, the coupling reaction is performed using dichloromethane as an organic solvent.

In some embodiments, step (b) includes removing the solvent, e.g., under reduced pressure at ambient temperature, and optionally substituting the counter-ion by contacting with an ion exchange resin.

In accordance with the present invention, the besylate anion can be introduced by contacting with an ion exchange resin containing besylate anions, e.g., by dissolving the cisatracurium salt (wherein an anion (X⁻) is other than besylate), e.g., in an organic solvent comprising an aliphatic alcohol, ketone or nitrile and contacting the ion exchange resin with the resulting solution. Suitable organic solvents that can be used in the ion exchange process include, e.g., methanol, ethanol, isopropanol, acetone, methyl ethyl ketone, acetonitrile and mixtures thereof. A preferred organic solvent for performing the ion exchange is methanol. In one embodiment, a solution containing the cisatracurium salt is applied to an ion exchange column carrying benzenesulfonate (besylate) anions, and cisatracurium besylate is removed from the column by eluting with an organic solvent. Suitable organic solvents that can be used for eluting cisatracurium besylate from such an ion exchange column include methanol, ethanol, isopropanol, acetone, methyl ethyl ketone, acetonitrile or a mixture thereof. A preferred elution solvent is methanol.

The purification process can be performed using any suitable purification method, e.g., filtration, extraction, precipitation, crystallization, slurrying or any suitable combination of these procedures. In one embodiment, the cisatracurium salt, e.g., cisatracurium besylate, is selectively precipitated by mixing the cisatracurium salt with a first solvent and adding a second organic solvent, or mixture of solvents, in which the cisatracurium salt is sparingly soluble to precipitate the cisatracurium salt as a purified product. The first solvent that can be used for precipitating cisatracurium besylate includes methanol, ethanol, n-propanol, isopropanol, acetone, methyl ethyl ketone, ethyl acetate, tetrahydrofuran, dichloromethane, chloroform or a mixture thereof. A particularly preferred first organic solvent for precipitating cisatracurium besylate is dichloromethane. Exemplary second organic solvents in which cisatracurium besylate is sparingly soluble include diethyl ether, isopropyl ether, tert-butyl methyl ether, toluene, 2-methyl-tetrahydrofuran (2-Me-THF), and C₅ to C₈ saturated hydrocarbons, such as, n-hexane, n-heptane, cyclohexane, and the like, and mixtures thereof. Preferred second organic solvents include mixtures of toluene and 2-Me-THF.

In accordance with the present invention, cisatracurium salts, e.g., cisatracurium besylate, also can be purified by slurrying in an organic solvent, optionally at an elevated temperature, and collecting cisatracurium besylate as a purified product. Exemplary organic solvents that can be used for purifying cisatracurium besylate by slurrying include ethyl acetate, toluene, tert-butyl methyl ether, diethyl ether, n-pentane, and mixtures thereof A particularly preferred organic solvent for purifying cisatracurium besylate by slurrying is n-pentane.

According to another embodiment, the removal of residual solvents from the cisatracurium besylate can be carried out by extracting with an organic solvent selected from

saturated hydrocarbons such as, e.g., n-pentane, n-hexane, cyclohexane, n-heptane, petroleum ether and the like, and mixtures thereof, preferably n-pentane, n-heptane and mixtures thereof. As the results presented in Table 3 show, extraction of aqueous acidic solution of cisatracurium can remove the residual solvents to a level, which is in accordance with the ICH guideline.
The ICH guideline is published by the “International Conference on Harmonization of Technical Requirements of Registration of Pharmaceuticals for Human Use (ICH).” According to this guidance (Appendixes 5-7: toxicological data for class 1-3 solvents respectively), the use of industrial solvents in active pharmaceutical ingredients is restricted according to their toxicity and safety features and a maximum allowable level is defined for each solvent accordingly.

The removal of residual solvents from the cisatracurium besylate can also be carried out by lyophilizing an aqueous acidic solution of the cisatracurium which contains t-butanol. As the results presented in Table 4 show, the content of residual solvents such as dichloromethane can be significantly reduced after the lyophilization.

The process of the present invention produces cisatracurium salts, e.g., cisatracurium besylate, in at least about 95% purity, preferably in at least about 98% purity, and more preferably in at least about 99.5% purity, as measured by HPLC. The process of the present invention produces cisatracurium salts, e.g., cisatracurium besylate, in an isomeric purity of at least about 97%, preferably in an isomeric purity of at least about 99% and more preferably in an isomeric purity of at least about 99.5%.

In accordance with the present invention, compounds (VIIA), (VIIB) and VIII can be used to synthesize cisatracurium besylate (I) without having to resort to a difficult and expensive HPLC purification or other conventional procedures, e.g., as described in U.S. '510.

### EXAMPLES

The following examples further illustrate the invention but should not be construed as in any way limiting its scope.

### Example 1

This example describes the preparation of cisatracurium besylate.

A mixture of (1R-cis)-1-[(3,4-dimethoxyphenyl)methyl]-1,2,3,4-tetrahydro-6,7-dimethoxy-2-methyl-2-carboxyethyl-isoquinolinium besylate (Compound VIIA wherein R is H) (hereinafter the “cis-acid besylate”) (1.18 g, 2 mmol), anhydrous 1,5-pentanediol (0.1 g, 0.96 mmol, 0.48 eq.), benzenesulfonic acid (0.175 g, 1.11 mmol, 0.56 eq.) and anhydrous CaSO₄ (2.0 g) in dichloromethane (20 ml) was stirred at ambient temperature for 23 hours to afford a reaction mixture containing 75.1% of cisatracurium besylate, 8.7% of (1R-cis)-1-[(3,4-dimethoxyphenyl)methyl]-1,2,3,4-tetrahydro-6,7-dimethoxy-2-methyl-2-[3-[(5-hydroxypentyl)oxy]-3-oxopropyl-isoquinolinium besylate, the monoester besylate (Compound VIII), and 16.2% of the cis-acid besylate starting material. Then, benzenesulfonic acid (0.05 g) and CaSO₄ (0.8 g) were added to the reaction mixture and the mixture was stirred at ambient temperature for additional 24 hours. According to an HPLC analysis the product contained: 89.2% of cisatracurium besylate, 3.2% of monoester besylate (Compound VIII) and 7.6% of cis-acid besylate. CaSO₄ was then collected by filtration and the filtrate was washed with water (3×15 ml). After phase separation, the organic phase was dried over MgSO₄ and dichloromethane was evaporated under reduced pressure to obtain a crude product as colorless foam. n-pentane (20 ml) was added to the foam and the mixture was stirred at ambient temperature for 2 hours to obtain a suspension. The solvent was evaporated under reduced pressure to afford crude cisatracurium besylate as colorless solid. Yield: 1.07 g, 89.6%, purity by HPLC: 98.2%; containing 1% of the monoester (Compound VIII) and 0.5% of the cis-acid besylate.

### Example 2

This example describes the preparation of cisatracurium besylate.

A mixture of the cis-acid besylate (1.0 g, 1.7 mmol), anhydrous 1,5-pentanediol (0.085 g, 0.82 mmol, 0.48 eq.), benzenesulfonic acid (0.3 g, 1.89 mmol, 1.1 eq.) and anhydrous CaSO₄ (2.0 g) in dichloromethane (20 ml) was stirred at ambient temperature for 23 hours to afford a reaction mixture containing 90.5% of cisatracurium besylate, 4.3% of monoester besylate (compound VIII) and 5.2% of cis-acid besylate. CaSO₄ was then collected by filtration and the filtrate was washed with water (3×30 ml). The phases were separated and the organic phase was dried over MgSO₄ and the dichloromethane was evaporated under reduced pressure to obtain a crude product as colorless foam. n-pentane (20 ml) was added to the foam and the mixture was stirred at ambient temperature for 2 hours to obtain a suspension. The solvent was evaporated under reduced pressure to afford crude cisatracurium as colorless solid. Yield: 0.95 g, 93.1%, purity by HPLC: 99.1%; containing 0.5% of the monoester (Compound VIII) and 0.1% of the cis-acid besylate.

### Example 3

This example describes the preparation of cisatracurium besylate.

A mixture of cis-acid besylate (5.0 g, 8.5 mmol), anhydrous 1,5-pentanediol (0.43 g, 4.13 mmol, 0.486 eq.), benzenesulfonic acid (1.5 g, 9.48 mmol, 1.1 eq.) and anhydrous CaSO₄ (12.5 g) in dichloromethane (100 ml) was stirred at ambient temperature for 24 hours to afford a reaction mixture containing 90.5% of cisatracurium besylate, 3.0% of monoester besylate and 6.5% of cis-acid besylate. CaSO₄ was then collected by filtration and the filtrate was washed with water (6×70 ml). The organic phase was dried over MgSO₄ and the dichloromethane was evaporated under reduced pressure to obtain a crude product as colorless foam. Toluene (50 ml) was added to the foam and the mixture was stirred at ambient temperature for half an hour and then the toluene was removed to dryness under reduced pressure to obtain a solid. n-pentane (50 ml) was added to the solid, the mixture was stirred at ambient temperature for 2 hours and the solvent was collected by decantation. A second portion of n-pentane (50 ml) was added to the solid and the n-pentane was evaporated under reduced pressure to afford crude cisatracurium as colorless solid. Yield: 4.4 g, 85.8%, purity by HPLC: 99.7%; containing 0.04% of monoester and 0.09% of cis-acid besylate.

### Example 4

This example describes the preparation of cisatracurium besylate.

The cis-acid besylate (0.53 g, 0.902 mmol) was dissolved in dichloromethane (10 ml). The solution was cooled to 0° C. and oxalyl chloride (0.086 ml, 0.992 mmol) was added in portions at 0° C. The temperature was allowed to reach room temperature and the reaction mixture was stirred at this temperature for 2 hours. The reaction mixture was then cooled to 0° C. and 1,5-pentanediol (0.050 ml, 0.473 mmol) was added in portions. The temperature was allowed to reach room temperature and the reaction mixture was stirred at this temperature for 4 hours. Then, the reaction mixture was concentrated to dryness under reduced pressure at room temperature to afford the crude acid chloride product. The residue was dissolved in a mixture of water (10 ml) and toluene (20 ml) to afford a two phase system and the layers were separated. The aqueous layer, containing the cisatracurium besylate, was washed with a mixture of ethyl acetate and n-heptane (5:1 v/v, 20 ml) followed by toluene (20 ml). The aqueous layer was back-extracted with dichloromethane (50 ml). The dichloromethane layer, containing the cisatracurium besylate, was dried over magnesium sulfate and evaporated under reduced pressure at 25° C. to afford crude cisatracurium besylate (0.260 g, 23% yield, HPLC purity: 61%).

### Example 5

This example describes the preparation of cisatracurium iodide.

“Acid iodide,” that is Compound VIIA wherein R is H, iodide (0.50 g, 0.917 mmol) was dissolved in dichloromethane (15 ml). The solution was cooled to 0° C. and thionyl chloride (0.10 ml, 1.376 mmol) was added in portions at 0° C. The reaction mixture was allowed to reach room temperature and stirred at room temperature for 2 hours. The mixture was cooled to 0° C. and 1,5-pentanediol (0.05 ml, 0.481 mmol) was added in portions. After the addition, the mixture was allowed to reach room temperature and stirred at this temperature for 14 hours. Then, the reaction mixture was concentrated to dryness under reduced pressure at room temperature to afford a semi-solid oil of crude cisatracurium iodide (0.752 g, 69% yield, HPLC purity: 80%, containing 10% of the starting material).

### Example 6

This example describes the preparation of cisatracurium besylate from the cis-ester besylate using the acidic catalyst NaHSO₄.SiO₂.

A mixture of (1R-cis)-1-[(3,4-dimethoxyphenyl)methyl]-1,2,3,4-tetrahydro-6,7-dimethoxy-2-methyl-2-tert-butoxycarbonylethyl-isoquinolinium besylate, that is Compound VIIA while R=tert-butoxycarbonyl (hereinafter the “cis-ester besylate”) (1.0 g, 1.56 mmoles), anhydrous 1,5-pentanediol (0.080 g, 0.78 mmoles, 0.5 eq) and anhydrous NaHSO₄.SiO₂ (0.15 g, 0.82 mmoles, 0.52 eq) was stirred in dichloromethane (10 ml) under reflux for 16 hours. According to the HPLC analysis, the reaction mixture contained 2.9% of cisatracurium besylate, 14.0% monoester besylate, 6.7% cis-acid besylate and 76.4% of the cis-ester besylate. Subsequently, an additional amount of NaHSO₄.SiO₂ (0.15 g, 0.82 mmoles, 0.52 eq.) was added and the mixture was stirred under reflux for further 18 hours. According to a sample of the reaction mixture that was withdrawn and injected to the HPLC, the mixture contained 32.0% of cisatracurium besylate, 17.5% of monoester besylate, 12.8% of cis-acid besylate and 37.3% of cis-ester besylate.

### Example 7

This example describes the preparation of cisatracurium besylate from the cis-ester besylate using the acidic catalyst Amberlyst®15 hydrogen form.

A mixture of the cis-ester besylate (1.0 g, 1.56 mmoles), anhydrous 1,5-pentanediol (0.080 g, 0.78 mmoles, 0.5 eq.) and dry Amberlyst® 15 hydrogen form (0.25 g) was stirred in dichloromethane (10 ml) at reflux for 8 hours. According to the HPLC analysis, the reaction mixture contained 3.2% of cisatracurium besylate, 6.1% of monoester besylate, 24.2% of cis-acid besylate and 66.6% of cis-ester besylate. Subsequently, the reaction mixture was stirred at ambient temperature for 7 days. A sample of the reaction mixture that was withdrawn and injected to the HPLC contained 21.9% of cisatracurium besylate, 10.0% of monoester besylate, 33.8% of cis-acid besylate and 34.4% of cis-ester besylate.

### Example 8

This example describes the preparation of cisatracurium besylate from the cis-acid besylate using the acidic catalyst NaHSO₄.SiO₂.

Method A: A mixture of cis-acid besylate (0.6 g, 1.02 mmoles), anhydrous 1,5-pentanediol (0.049 g, 0.51 mmoles, 0.5 eq.) and NaHSO₄.SiO₂ (0.13 g, 0.71 mmoles, 0.7 eq.) was stirred in dichloromethane (10 ml) at ambient temperature for 2 days. According to the HPLC analysis, the reaction mixture contained 30.2% of cisatracurium besylate, 37.0% of monoester besylate, and 32.8% of cis-acid besylate. Subsequently, the reaction mixture was stirred under reflux for 6 hours. According to a second HPLC analysis, the mixture contained 49.8% of cisatracurium besylate, 34.7% of monoester besylate, and 15.5% of cis-acid besylate. The mixture was allowed to cool to ambient temperature and additional amount of NaHSO₄.SiO₂ (0.13 g, 0.71 mmoles, 0.7 eq.) was added. The reaction mixture was stirred at ambient temperature for about 16 hours. According to a third HPLC analysis, the mixture contained 55.0% of cisatracurium besylate, 32.4% of monoester besylate and 12.6% of cis-acid besylate. The mixture was stirred under reflux for an additional 4 hours. According to a fourth sample of the reaction mixture that was withdrawn and injected to the HPLC, the mixture contained 62.4% of cisatracurium besylate, 30.8% of monoester besylate, and 6.8% of cis-acid besylate.

Method B: A mixture of cis-acid besylate (0.5 g, 0.85 mmoles), anhydrous 1,5-pentanediol (0.041 g, 0.425 mmoles, 0.5 eq.), NaHSO₄.SiO₂ (0.34 g, 1.85 mmoles, 2.18 eq.) and magnesium sulfate (0.25 g) was stirred in dichloromethane (10 ml) at ambient temperature for 24 hours. According to the HPLC analysis, the mixture contained 40.1% of cisatracurium besylate, 12.1% of monoester besylate and 47.5% of the cis-acid besylate. Subsequently, NaHSO₄.SiO₂ (0.34 g, 1.85 mmoles, 2.18 eq.), anhydrous magnesium sulfate (0.25 g) and benzenesulfonic acid dihydrate (0.02 g) were added and the mixture was stirred at ambient temperature for 3.5 hours. According to the second HPLC analysis, the mixture contained 49.9% of cisatracurium besylate, 10.7% of monoester besylate and 38.7% of cis-acid besylate.

Method C: A mixture of cis-acid besylate (0.6 g, 1.02 mmoles), anhydrous 1,5-pentanediol (0.049 g, 0.51 mmoles, 0.5 eq.), NaHSO₄.SiO₂ (0.34 g, 1.85 mmoles, 1.81 eq.) was stirred in dichloromethane (10 ml) under reflux for 3 hours. According to the HPLC analysis, the mixture contained 23.7% of cisatracurium besylate, 9.5% of monoester besylate and 66.7% of cis-acid besylate. Subsequently, the mixture was stirred overnight at ambient temperature. According to the second HPLC analysis, the mixture contained 47.6% of cisatracurium besylate, 6.6% of monoester besylate and 46.0% of cis-acid besylate. The reaction was then stirred under reflux for 7 hours to afford a mixture which according to the HPLC analysis contained 54.9% of cisatracurium besylate, 2.0% of monoester besylate and 43.0% of cis-acid besylate.

### Example 9

This example describes the preparation of cisatracurium besylate from the cis-acid besylate using the acidic catalyst Amberlyst®15 hydrogen form.

A mixture of the cis-acid besylate (0.5 g, 0.85 mmoles), anhydrous 1,5-pentanediol (0.041 g, 0.425 mmoles, 0.5 eq.), dry Amberlyst®15 hydrogen form (0.25 g) was stirred in dichloromethane (10 ml) under reflux for 8 hours. According to the HPLC analysis, the reaction mixture contained 19.3% of cisatracurium besylate, 2.1% of monoester besylate and 78.6% of the cis-acid besylate. The mixture was stirred at ambient temperature for about 16 hours. According to a second HPLC analysis, the mixture contained 21.4% of cisatracurium besylate, 2.2% of monoester besylate and 76.4% of cis-acid besylate. An additional portion of Amberlyst®15 hydrogen form (0.25 g) and anhydrous magnesium sulfate (0.2 g) were added and the reaction mixture was stirred at ambient temperature for 20 hours. According to a third HPLC analysis, the mixture contained 36.8% of cisatracurium besylate, 2.0% of monoester besylate and 61.1% of cis-acid besylate.

### Example 10

This example describes the preparation of cisatracurium besylate from the cis-acid besylate using benzenesulfonic acid and silica gel (pH 3.0-5.0).

A mixture of the cis-acid besylate (0.5 g, 0.85 mmoles), anhydrous 1,5-pentanediol (0.041 g, 0.425 mmoles, 0.5 eq.), dry silica gel of pH 3.0-5.0 (0.6 g) was stirred in dichloromethane (10 ml) at ambient temperature for 18 hours. According to an HPLC analysis, the mixture contained 14.4% of cisatracurium besylate, 4.8% of monoester besylate and 80.8% of cis-acid besylate. Subsequently, anhydrous magnesium sulfate (0.25 g) was added and the reaction mixture was stirred at ambient temperature for 50 hours. According to a second HPLC analysis, the mixture contained 47.9% of cisatracurium besylate, 3.9% of monoester besylate and 48.2% of cis-acid besylate.

### Example 11

This example describes the preparation of cisatracurium besylate from the cis-acid besylate in presence of benzenesulfonic acid in dichloromethane and purification of the obtained cisatracurium besylate by precipitation.

A reaction vessel, equipped with mechanical stirrer and thermometer, was charged with the 1,5-pentanediol (0.083 g, 8 mmole) under constant mixing, and benzenesulfonic acid was added (1.26 g, 8 mmole). Then, dichloromethane was added (200 ml) and stirring was maintained at 25° C. for 10 minutes to obtain a suspension. Cis-acid besylate was added (12.47 g, 21.2 mmole). The mixture was heated to reflux (T_{bath}=60° C.) and stirring was maintained for 12 hours while removing the solvent by azeotropic distillation using Dean Stark apparatus. According to a sample which was withdrawn and checked by HPLC, the cisatracurium besylate content at the end of the reaction was 95.2%.

The solution, containing the product, was evaporated to a reduced volume of 68 ml, and toluene was added (100 ml). Stirring was maintained at 25° C. for 30 minutes and 2-MeTHF was added (200 ml). Stirring was maintained at 25° C. for 45 minutes during which time a solid was formed. The solvents were removed by decantation and the thus obtained residue was dissolved in dichloromethane (30 ml). The dichloromethane solution was evaporated in vacuum at a temperature of about 30° C. to obtain 9.2 g cisatracurium besylate in 92% yield, having purity of 97.6%. Repeating the precipitation process afforded a product having purity of 98.2%.

### Example 12

This example describes the preparation of Compound VIII.

Cis-acid besylate (0.78 g, 1.327 mmol) was dissolved in dichloromethane (3 ml). The solution was cooled to 10°-15° C. and oxalyl chloride (0.171 ml, 1.991 mmol, 1.5 about 16 hours. The reaction mixture was concentrated under reduced pressure to remove the dichloromethane and excess oxalyl chloride. The remaining residue containing compound (VIIB) was dissolved in dry tetrahydrofuran (THF) (2 ml) and the resulting solution was added dropwise during about 30 minutes to 1,5-pentanediol (1.4 ml, 13.27 mmoles, 10 eq.) and dry THF (3 ml). The reaction mixture was stirred at 10°-15° C. for 2 hours. The reaction mixture was concentrated under reduced pressure to remove the THF. Water (20 ml) and toluene (20 ml) were added to the remaining residue and the mixture was stirred at ambient temperature to afford two layers. The layers were separated and the aqueous layer was washed with a mixture of ethyl acetate and n-heptane (4:1 v/v, 20 ml). Dichloromethane (80 ml) was added to the aqueous layer and the mixture was stirred at ambient temperature in order to extract the product into the organic layer. The layers were separated and the dichloromethane layer was dried over magnesium sulfate and evaporated under reduced pressure at ambient temperature to afford compound (VIII) (0.020 g, 2% yield, HPLC purity: 88%).

### Example 13

This example describes the preparation of Compound VIII.

“Acid iodide,” (Compound VIIA wherein R is H, and X⁻ is iodide) (0.5 g, 0.917 mmoles) was dissolved in dichloromethane (15 ml). The mixture was cooled to 0° C. and thionyl chloride (100 μL, 1.376 mmoles, 1.5 eq.) was added dropwise at 0° C. to the resulting suspension. The mixture was stirred at ambient temperature for 2 hours. Subsequently, 1,5-pentanediol (578 μL, 5.502 mmoles, 6 eq.) was added dropwise at 0° C. and the mixture was stirred at 25° C. for 14 hours. Then, the reaction mixture was concentrated under reduced pressure to afford an oil. The oil was dissolved in dichloromethane (100 ml) and a pH 4 buffer, consisting of citric acid, sodium hydroxide and sodium chloride, was added (15 ml) followed by addition of an aqueous saturated solution of sodium thiosulfate (5 ml) to afford a two phase system. The layers were separated, the organic layer was dried over magnesium sulfate and the dichloromethane was removed under reduced pressure to afford a pale yellow solid (0.43 g, 73% yield). The residual solid was dissolved in a minimal amount of dichloromethane. Diethyl ether was added to the resulting solution drop wise to afford a white suspension. A precipitate was collected by filtration and washed with diethyl ether to afford a white solid (HPLC: 87% compound (VIII), 6% compound (VIIA), 6% cisatracurium).

### Example 14

This example describes the preparation of cisatracurium besylate from Compound VIII.

Monoester besylate (Compound VIII) (173.5 mg, 0.275 moles) was dissolved in dichloromethane (15 ml). Benzenesulfonic acid (50 mg, 1 eq.) was added to this solution followed by addition of CaSO₄ (333 mg). The thus formed suspension was stirred for 2 minutes and cis-acid besylate (450 mg, 1 eq.) was added in one portion. The reaction mixture was stirred for 5 days after which time a sample was withdrawn and injected to the HPLC showing that the reaction mixture contained 59% of cisatracurium besylate.

### Example 15

This example describes purification of cisatracurium besylate by extraction of the residual solvents with pentane or heptane.

A reaction vessel equipped with mechanical stirrer and thermometer was charged with cisatracurium besylate (10.0 g) and cold aqueous acidic solution (pH=4.0 with benzenesulfonic acid) (300 ml). The mixture was stirred at 10° C. to obtain a solution, which was extracted eight times with cold n-pentane (800 ml each extraction). The layers were separated and the aqueous phase, containing the product, was lyophilized. The method described herein was repeated using n-heptate. Table 3 specifies the quantites of residual solvents in the samples of cisatracurium besylate before and after the extractions with n-pentane and n-heptane.

TABLE 3

| | | Quantity of the solvent | Quantity of the solvent | Quantity of the solvent |
|---|---|---|---|---|
| | | before n-pentane or n- | after n-pentane extraction | after n-heptane extraction |
| Solvent | ICH limit, ppm | hexane extraction, ppm | and lyophilization, ppm | and lyophilization, ppm |
| n-pentane | 5000 | NC | 504 | NC |
| n-heptane | 5000 | NC | NC | 183 |
| Dichloromethane | 600 | 100 | 36 | 0 |
| 2-Me-THF* | — | 17486 | 290 | 173 |
| Toluene | 900 | 7506 | 37 | 21 |

ND = not detected. NC = not checked (because the solvent is not supposed to be contained in this sample).
*2-methyl-tetrahydrofuran (2-Me-THF) is not mentioned in the ICH guideline.

### Example 16

This example describes the purification of the cisatracurium besylate from residual solvents by lyophilization with t-butanol and water.

A reaction vessel, equipped with mechanical stirrer and thermometer, was charged with cisatracurium besylate (10.0 g), cold acidified aqueous solution (pH=4.0 with benzenesulfonic acid) (300 ml) and t-butanol (60 ml). Stirring was applied for 15 minutes to obtain a solution, which was lyophilized for about 20 hours to afford 9.0 g of cisatracurium besylate, in 90% yield. Table 4 details the content of residual solvents in the cisatracurium besylate before and after lyophilization with t-butanol.

TABLE 4

| | | Quantity of the solvent | Quantity of the solvent | Quantity of the solvent |
|---|---|---|---|---|
| | | before lyophilizing with | after lyophilizing with | after lyophilizing with |
| Solvent | ICH limit, ppm | H₂O:t-BuOH, ppm | H₂O:t-BuOH 1:1, ppm | H₂O:t-BuOH 4.5:1, ppm |
| Dichloromethane | 600 | 100 | 0 | 0 |
| 2-Me-THF* | — | 16,130 | 35 | 71 |
| Toluene | 900 | 27,380 | 290 | 615 |

*2-methyl-tetrahydrofuran (2-Me-THF) is not mentioned in the ICH guideline.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing a cisatracurium salt, the process comprising:
(a) reacting a compound of formula (VIIA): 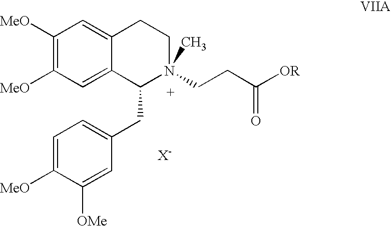
wherein X⁻ is iodide or besylate and R is H or a C₁-C₆ alkyl (methyl, ethyl, n-propyl, isopropyl, butyl, isobutyl, sec-butyl, tert-butyl, pentyl or hexyl), with 1,5-pentanediol to produce a cisatracurium salt, or reacting a compound of formula (VIIA) with 1,5-pentanediol to form an intermediate compound of formula (VIII): 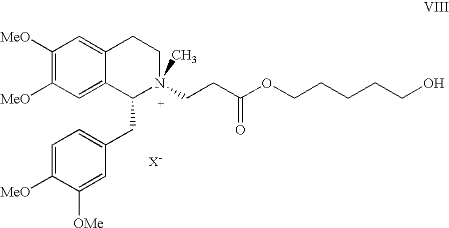
and reacting the compound of formula (VIII) with a compound of formula (VIIA) to produce a cisatracurium salt, wherein X⁻ and R are as defined herein;
(b) optionally performing an ion exchange;
(c) isolating the cisatracurium salt; and
(d) purifying the cisatracurium salt.

2. The process of claim 1, wherein step (a) comprises reacting compound (VIIA) or compound (VIII) with 1,5-pentanediol in the presence of an acidic catalyst, optionally with removal of water, to form a cisatracurium salt.

3. The process of claim 2, wherein water is removed by a method selected from azeotropic distillation, adding a drying agent, and combinations thereof.

4. The process of claim 2, wherein the reaction of compound (VIIA) or (VIII) with 1,5-pentanediol is carried out in the presence of an organic solvent, which is toluene, and the acid is sulfuric acid, or the reaction is carried out in the presence of an organic solvent, which solvent is dichloromethane, and the acid is benzenesulfonic acid.

5. The process of claim 1, wherein step (a) comprises:
(i) reacting compound (VIIA), wherein R is H, with an activating agent to form a compound of the formula VIIB: 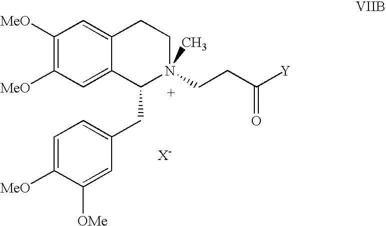
wherein Y is halogen, OR₁, or OCOR₁, wherein R₁ is C₁-C₆ alkyl and r is iodide or besylate; and
(ii) reacting the activated compound (VIIB) with 1,5-pentanediol to produce a cisatracurium salt, or reacting the activated compound (VIIB) with 1,5-pentanediol to form intermediate compound (VIII), and reacting compound (VIII) with compound (VIIB) or (VIIA) to produce a cisatracurium salt.

6. The process of claim 5, wherein the activating agent is a chlorinating agent, an esterifying agent or an acid anhydride forming agent.

7. The process of claim 6, wherein the activating agent is a chlorinating agent selected from phosphorus oxychloride, thionyl chloride, phosphorous pentachloride and oxalyl chloride.

8. The process of claim 5, wherein the reaction of compound (VIIA) with an activating agent is carried out in the presence of an organic solvent, which is dichloromethane, chloroform, 1,2-dichloroethane, xylenes, toluene or a mixture thereof.

9. The process of claim 8, wherein the organic solvent is dichloromethane.

10. The process of claim 7, wherein the chlorinating agent is present in a range of from about 1.0 equivalent to about 2.0 equivalents relative to compound (VIIA).

11. The process of claim 5, wherein the 1,5-pentanediol is added to the activated compound (VIIB) gradually at a temperature of less than about 20° C.

12. The process of claim 5, wherein the 1,5-pentanediol is present in a range of from about 0.5 equivalents to about 1.0 equivalents relative to compound (VIIB).

13. The process of claim 3, wherein the acidic catalyst is selected from CaSO₄/benzenesulfonic acid, NaHSO₄.SiO₂, Amberlyst®15 and a mixture of benzenesulfonic acid and silica gel of pH 3.0-5.0.

14. The process of claim 1, wherein the ion exchange step is performed and comprises contacting the cisatracurium salt with an ion exchange resin carrying besylate anions, to produce cisatracurium besylate, wherein at least one anion in the starting cisatracurium salt is not a besylate anion.

15. The process of claim 1, wherein the cisatracurium salt produced in step (a) or (b) is cisatracurium besylate, and step (d) comprises purifying the cisatracurium besylate by a method selected from filtration, extraction, precipitation, crystallization, slurrying, and combinations thereof.

16. The process of claim 15, wherein step (d) comprises mixing the cisatracurium besylate produced in step (a) or (b) with a first solvent and adding a second organic solvent or mixture of solvents, in which the cisatracurium besylate is sparingly soluble, to precipitate the cisatracurium besylate as a purified product.

17. The process of claim 16, wherein the first solvent is methanol, ethanol, n-propanol, isopropanol, acetone, methyl ethyl ketone, ethyl acetate, tetrahydrofuran, dichloromethane, chloroform or a mixture thereof.

18. The process of claim 17, wherein the first organic solvent is dichloromethane.

19. The process of claim 16, wherein, the second organic solvent or mixture of solvents is diethyl ether, isopropyl ether, tert-butyl methyl ether, toluene, 2-methyl-tetrahydrofuran (2-Me-THF), n-hexane, n-heptane, cyclohexane, petroleum ether, or a mixture thereof.

20. The process of claim 19, wherein the second organic solvent is a mixture of toluene and 2-Me-THF.

21. The process of claim 15, wherein the cisatracurium besylate is purified by slurrying in an organic solvent, optionally at an elevated temperature, and collecting cisatracurium besylate as a purified product.

22. The process of claim 21, wherein the organic solvent used for purifying cisatracurium besylate by slurrying is ethyl acetate, toluene, tert-butyl methyl ether, diethyl ether, n-pentane, or a mixture thereof.

23. The process of claim 22, wherein the organic solvent for purifying cisatracurium besylate by slurrying is n-pentane.

24. The process of claim 15, comprising removing residual solvents from the cisatracurium besylate by extraction with an organic solvent selected from n-pentane, n-hexane, cyclohexane, n-heptane, petroleum ether and mixtures thereof.

25. The process of claim 24, wherein the organic solvent for removing residual solvents is n-pentane or n-heptane.
